(19) United States
(12) Patent Application Publication
(10) Pub. No.: US 2020/0216464 A1
(43) Pub. Date: Jul. 9, 2020

(54) NEW COMPOUND USEFUL IN THE MANUFACTURE OF MEDICAMENTS

(71) Applicant: Hoffmann-La Roche Inc., Little Falls, NJ (US)

(72) Inventors: Paul SPURR, Basel (CH); Roland AGRA, Basel (CH)

(73) Assignee: Hoffmann-La Roche Inc., Little Falls, NJ (US)

(21) Appl. No.: 16/818,903

(22) Filed: Mar. 13, 2020

Related U.S. Application Data
(63) Continuation of application No. PCT/EP2018/074386, filed on Sep. 11, 2018.

(30) Foreign Application Priority Data
Sep. 14, 2017 (EP) 17191116.7

(51) Int. Cl. C07D 495/14

(52) U.S. Cl. CPC C07D 495/14

(57) ABSTRACT

The present invention relates to a compound of formula (I) as defined in the description and in the claims. The compound of formula (I) can be used in the manufacture of medicaments.

### CROSS REFERENCE TO RELATED APPLICATIONS

This application is a continuation of International Application No. PCT/EP2018/074386 having an international filing date of Sep. 11, 2018, and which claims benefit under 35 U.S.C. § 119 to European Patent Application No. 17191116.7, filed Sep. 14, 2017; all of which are incorporated by reference in their entirety.

### FIELD OF INVENTION

The present invention relates to a new compound which is useful in the manufacture of medicaments. The invention relates in particular to a compound of formula (I)

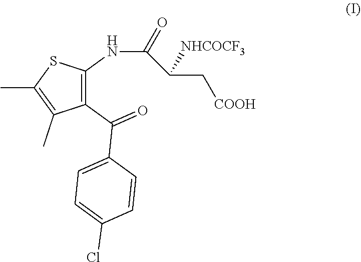

and to a process for its manufacture.

The compound of formula (I) is particularly advantageous in that is gives an easy and convenient access to the compound of formula (IX)

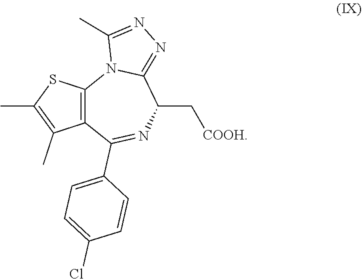 [SMILES: Cc1sc2c(c1C)C(c1ccc(Cl)cc1)=N[C@@H](CC(=O)O)c1nnc(C)n1-2]

The compound of formula (IX) is a key intemediate in the synthesis of several useful pharmaceutically active compounds, including for example the molecule known as JQ1.

The known syntheses of the compound of formula (IX) however involve many steps, sometimes with limited yields and, due to selectivity issues and requirements, necessitate the use of expensive starting materials and reagents.

The above problems have been resolved by the provision of the compound and process of the invention.

According to the process of the invention, the compound of formula (I) is thus surprisingly obtained from the reaction of the compound of formula (II) with the compound of formula (III) as the major product, although the compound of formula (III) is a poor nucleophile. Its amino group has a reduced reactivity due to steric hindrance and the electron delocalization on the carbonyl through the thiophene ring.

The compound of formula (III) reacts regioselectively with the compound of formula (II) without the presence of an activating agent. The CF₃CO— group both protects the amino on the compound of formula (II) and contributes to the regioselectivity due to its electron withrawing properties.

The compound of formula (II) is conveniently obtained from (S)-aspartic acid (IV), a cheap, commercialy available precursor, through a single step that achieves the activation of the electrophilic site and the protection of the amino group.

The compounds of formula (I) and (V) can for example be prepared according to Scheme 1.

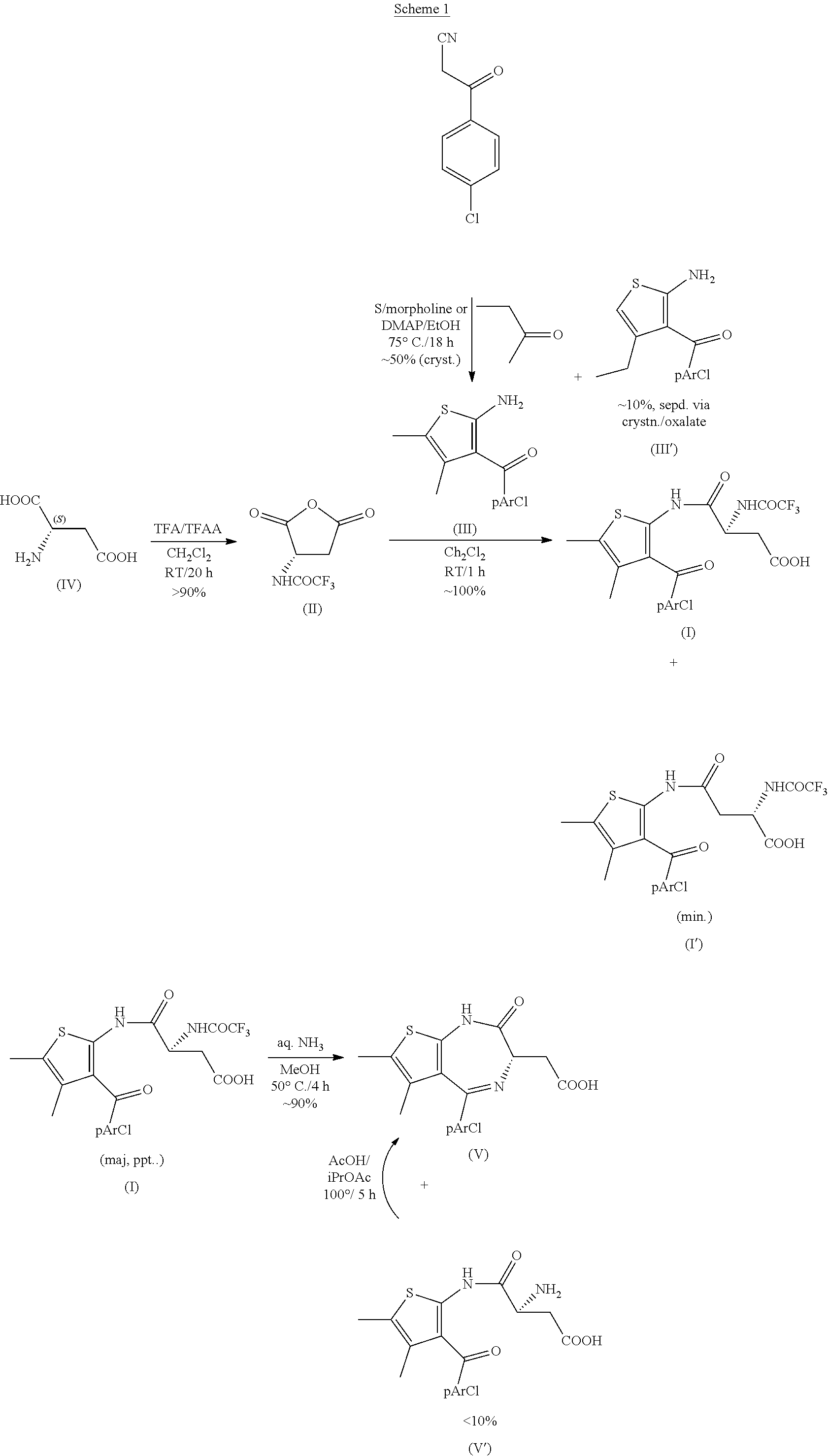

The compound of formula (III) can be prepared by known methods, for example by the reaction of 3-(4-chlorophenyl)-3-oxopropionitrile in the presence of butan-2-one, sulfur and a base to arrive at the compound of formula (III). The minor undesired isomer (III′) can be removed through crystallization of the oxalate salt (WO 2018/109053).

Acylation of aminothiophene (III) with anhydride (II) produces the desired regioisomer (I) as the major product which can be precipitated from the reaction mixture through the addition of an antisolvent such as heptane. The minor isomer (I′) remains in the mother liquor to a large extent. By this means, the original reaction product ratio of ca. 5:1 (I/I′) can be enriched to ˜7:1 in the isolated material. Although racemization can be an issue with reactions of activated aminoacids, the S/R ratio of the recovered product (1) is high (99:1). Catalysis with a wide variety of Lewis or Bronstead acids failed to improve the regioselectivity, however the influence of the solvent type is significant. Best ratios of I/I′ are attained in non-polar solvents, particularly CH₂Cl₂ or toluene. Temperature has a lesser effect on the outcome but an optimal regio- and enantio-selectivity outcome is acquired between 0° C.-RT. Below 0° C. the reaction rate is very slow. The nature of the protecting group is decisive for the regioselectivity—the higher the electron withdrawing ability, the better the ratio I/I′. Substituents such as MeCO, HCO, BOC and BnOCO all render lower selectivities. Perfluoroacyl-groups did not induce any improvement over CF₃CO and contrary to expectation, the unprotected aminoanhydride (as the HCl-salt) did not undergo the condensation effectively.

Not only does the CF₃CO-group confer the desired reaction selectivity, compound (II) can readily be prepared in a single step from (S)-aspartic acid in excellent yield without racemization and the protecting group can easily be removed later in the synthesis, again without racemization. Other derivatives of the anhydride (II) typically are prepared in two steps and their formation as well as deprotection thereafter can be less straight forward. The enriched material (I) isolated directly from the reaction mixture can be carried on into the next step.

Deprotection of intermediate (I) is effected with methanolic ammonia or more efficiently with aqueous NH₃ in MeOH at reflux. Concommitant ring formation occurs under the reaction conditions, yielding the acid (V) as the major product. A small amount (<10%) of the open form (V′) nevertheless remains which can be separated to a certain amount by extraction and/or converted to the imine (V) under acidic conditions however with some racemization. The deprotected regiosomer arising from residual (I′) forms much slower and remains in the aqueous phase. Intermediate (V′) also converts to the ester (VI) under the ensuing reaction conditions.

The compound of formula (V) can be further reacted to arrive at the compound of formula (IX) according to the following Scheme 2. This follows in an analogous manner to that described previously for the tBu-ester of (VI) (WO 2018/109053, Tetrahedron Letters 2015, 56, 3454-3457; WO 2015/131113; Nature 2010, 468, 1067-1073) whereby the lactam is activated, acylated with acetylhydrazide and the intermediate (VII) cyclized to the triazole (VIII). Hydrolysis of the individual esters represented by formula (VIII) produces the acid (IX).

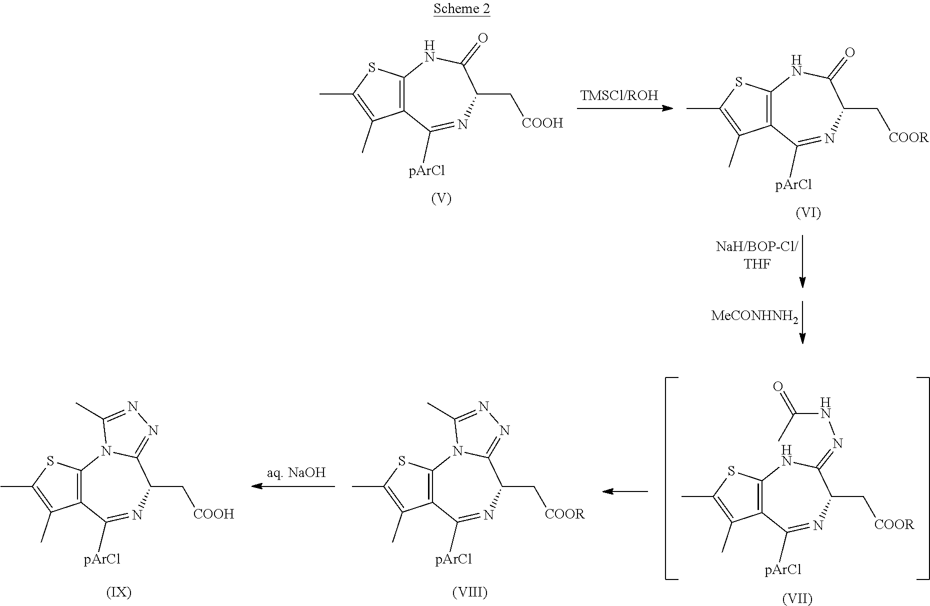

In scheme 2, R is alkyl, like e.g. Me, Et, iPr or iBu, preferably iBu.

In the present description, “room temperature” can for example be around 20° C.

The invention thus further relates to:

A process for the manufacture of a compound of formula (I) as defined above, comprising the reaction of a compound of formula (II)

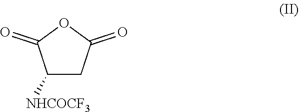

with a compound of formula (III)

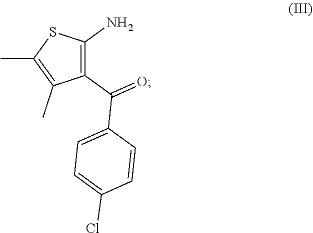 [SMILES: Cc1sc(N)c(C(=O)c2ccc(Cl)cc2)c1C]

A process as defined above, wherein the reaction of the compound of formula (II) with the compound of formula (III) is carried out in a suitable, preferably non-polar solvent;

A process as defined above, wherein the suitable solvent is selected from acetone, trifluoroethanol, acetonitrile, tetrahydrofuran, methyltetrahydrofuran, ethyl acetate, dichloromethane, t-butylmethylether, toluene, benzotrifluoride and heptane, in particular in ethyl acetate, dichloromethane, t-butylmethylether, toluene, benzotrifluoride and heptane.

A process as defined above, wherein the non-polar solvent is dichloromethane or toluene, in particular dichloromethane;

A process as defined above, wherein the reaction is carried out at a temperature between around 0° C. and around room temperature;

A process as defined above, wherein the compound of formula (II) is prepared by the reaction of a compound of formula (IV)

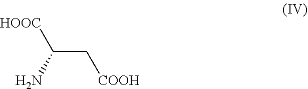

with trifluororoacetic anhydride;

A process for the manufacture of a compound of formula (V)

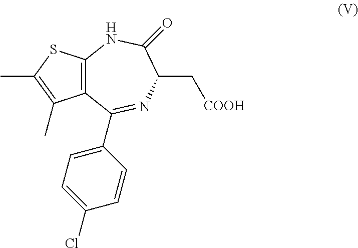 [SMILES: Cc1sc2c(c1C)C(c1ccc(Cl)cc1)=N[C@@H](CC(=O)O)C(=O)N2]

comprising the deprotection of the amino group —NHCOCF₃ of the compound of formula (I) into a primary amino group —NH₂ and concommitant ring formation to arrive at the compound of formula (V);

A process as defined above, wherein the deprotection of the amino group —NHCOCF₃ of the compound of formula (I) into a primary amino group —NH₂ is performed by reaction of the compound of formula (I) with a base in alcoholic medium, i.e. a medium comprising an alcohol and optionally water;

A process as defined above wherein the base is an amine, for example MeNH₂, Me₂NH, EtNH₂, Et₂NH, pyrrolidine, piperidine, or morpholine, or a metal hydroxide or carbonate, for example a Group I metal hydroxide or carbonate like for example Li, Na, K, Rb, Cs, Mg, Ca, Sr or Ba hydroxide or carbonate;

A process as defined above wherein the alcoholic medium comprises methanol, ethanol, n-propanol, i-propanol, n-butanol, i-butanol, s-butanol or t-butanol;

A process as defined above wherein the deprotection of the amino group —NHCOCF₃ of the compound of formula (I) into a primary amino group —NH₂ is effected by reaction of the compound of formula (I) with methanolic ammonia or aqueous ammonia in methanol, in particular with aqueous ammonia in methanol;

A process as defined above, wherein the deprotection of the amino group —NHCOCF₃ of the compound of formula (I) into a primary amino group and concommitant ring formation are accomplished at a temperature between around room temperature and around 100° C.;

A process as defined above, wherein the uncyclized side product of formula (V′)

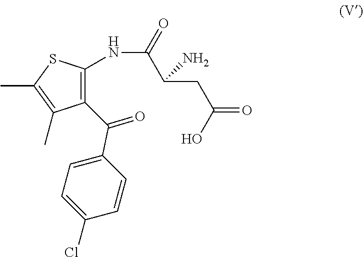 [SMILES: Cc1sc(NC(=O)[C@@H](N)CC(=O)O)c(C(=O)c2ccc(Cl)cc2)c1C]

that is obtained during the deprotection of the amino group —NHCOCF₃ of the compound of formula (I) into a primary amino group —NH₂, is separated from the crude reaction product and reacted with an acid to arrive at the compound of formula (V);

A process as defined above, wherein the acid reacted with the compound of formula (V′) is acetic acid, formic acid or a sulfonic acid, like for example methane sulfonic acid or paratoluene sulfonic acid, in particular acetic acid;

A process as defined above, wherein the compound of formula (V′) is reacted with the acid in toluene or isopropyl acetate, in particular in isopropyl acetate as described in WO 2018/109053; and

The use of a compound of formula (I) in the manufacture of a compound of formula (IX)

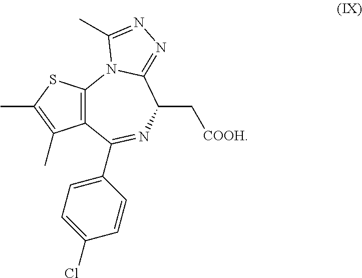 [SMILES: Cc1sc2c(c1C)C(c1ccc(Cl)cc1)=N[C@@H](CC(=O)O)c1nnc(C)n1-2]

The process as defined above, wherein the compound of formula (II) is prepared by the reaction of a compound of formula (IV) with trifluororoacetic anhydride can advantageously be performed by an adaptation of a known process (Chemische Berichte 1965, 98, 72-82, & WO 99/15494) wherein the trifluoroa.cetic acid solvent could be replaced to a large extent by dichloromethane and the product isolated by direct filtration.

It was found that the synthesis of the compound of formula (IX) as described in WO 2018/109053 suffered various degrees of ee-erosion and/or formation of a side product (VII′) in the cases of the Me, Et and iPr esters but not with the t-butyl ester. An efficient synthesis of the tBu ester from (V) could not be realized in this present case. However, we found surprisingly that both of the issues could be solved through employment of the iBu ester instead. As such, utilizing this ester avoided the formation of (VII′) which was formed in up to 10% in the case of the lowest ester (VI) (R=Me). This side product arises via an alternative ring closure pathway from intermediate (VII).

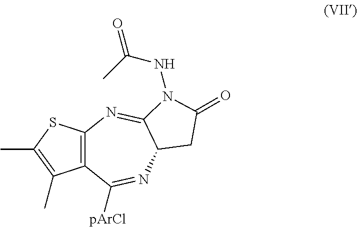

The invention thus also relates to a process for the manufacture of a compound of formula (IX), comprising:
(a) The reaction of a compound of formula (V) with i-BuOH under acid catalysis to arrive at a compound of formula (VI′)

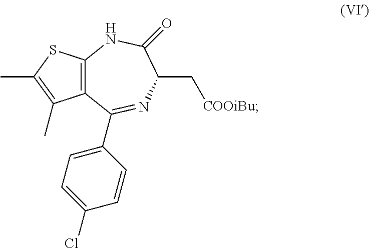

(b) The reaction of a compound of formula (VI′) with diethyl chlorophosphate, diphenyl chlorophosphate or bis(2-oxo-3-oxazolidinyl)phosphinic chloride and a base;
(c) The reaction of the product of step (b) with acetyl hydrazide followed by heating above room temperature to arrive at a compound of formula (VIII′)

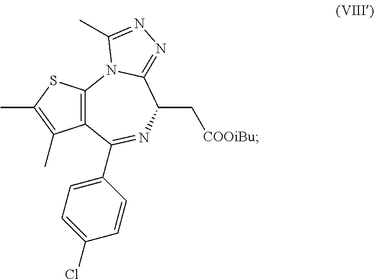

and
(d) The deprotection of the carboxyl group of the compound of formula (VIII′) to arrive at the compound of formula (IX) as defined above.

In step (a), acid catalysis can be advantageously effected with trimethylsilyl chloride (TMSCl).

Step (b) can be done at a temperature between e.g. −78° C. and room temperature.

In step (c), the reaction of the product of step (b) with acetyl hydrazide can advantageously be done at a temperature between −78° C. and 20° C.

The heating of step (c) above room temperature can advantageously be done at a temperature between 25° C. and 100° C. It forces the reaction to go to completion with no racemization being observed.

The product of step (b) can be used in step (c) as a crude product.

The product of step (c) can be used in step (d) as a crude product.

The compound of formula (IX) can advantageously be obtained without isolating or purifiying the intermediate products formed after steps (b) and (c).

The base of step (b) can advantageously be potassium tert.-pentoxide, potassium tert.-butoxide, sodium hydride, lithium tert.-pentoxide, lithium tert.-butoxide, sodium tert.-pentoxide or sodium tert.-butoxide more particularly sodium hydride.

In step (d), the deprotection of the carboxyl group of the compound of formula (VIII′) consists in hydrolysing the iBu-ester to create the acid (IX).

Step (d) can be performed by reacting the product of step (c) with a base in a protic medium.

The base of step (d) can advantageously be sodium hydroxide, in particular in a solvent like methanol or methanol/water mixtures.

LiOH and Cs₂CO₃ can also be used in step (d).

Step (d) can for example advantageously be performed by reacting the product of step (c) with sodium hydroxide in a mixture of water and methanol.

The compound of formula (IX) can for example be isolated after step (d) by crystallization from a mixture of isopropanol and n-heptane.

The invention will now be illustrated by the following examples which have no limiting character.

### EXAMPLES

### Example 1: N-((S)-2,5-Dioxotetrahydrofuran-3-yl)-2,2,2-trifluoroacetamide (II)

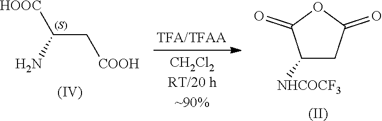

(5)-Aspartic acid (IV) (4.0 g, 30 mmol) was suspended with stirring in dichloromethane (15 ml) and trifluoracetic acid (2.6 ml, 33 mmol) was added. The mixture was cooled to 0-5° C. and trifluoroacetic anhydride (12.6 ml, 90 mmol) was added over five minutes. The reaction medium was brought to ambient temperature and stirred for 16 h. A thick white suspension formed which was diluted with dichloromethane (10 ml) and filtered. The residue was rinsed with additional dichloromethane then dried at 45° C./25 mb for 6 h; yield 5.6 g white crystalline solid (˜90%).

### Example 2: (S)-N-[3-(4-Chlorobenzoyl)-4,5-dimethylthiophen-2-yl]-3-(2,2,2-trifluoroacetylamino)-succinamic acid (I)

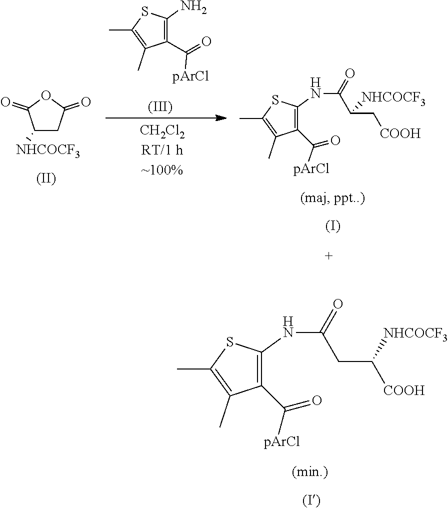

Aminothiophene (III) (1.9 g, 7 mmol) and anhydride (II) (1.6 g, 7.7 mmol) were suspended in dichloromethane (15 ml). On stirring the mixture for 0.25 h, a dark red solution arose and the reaction was complete after 1 h. Heptane (25 ml) was added and the yellow-orange suspension that formed was filtered and washed with 9:1 heptane-dichloromethane (20 ml). The product was dried at 45° C./25mb for 4 h; yield 2.8 g yellow crystalline solid (˜85%), HPLC: 82% (I)+13% (I′) in which I consisted of 99:1 S/R.

Scale-up to 20 mmol proceeding in a similar manner yielded almost quantitatively a product containing by HPLC 81% (I) and 14% (I′).

### Example 3: [(S)-5-(4-Chlorophenyl)-6,7-dimethyl-2-oxo-2,3-dihydro-1H-thieno[2,3-e][1,4]diazepin-3-yl]-acetic acid (V)

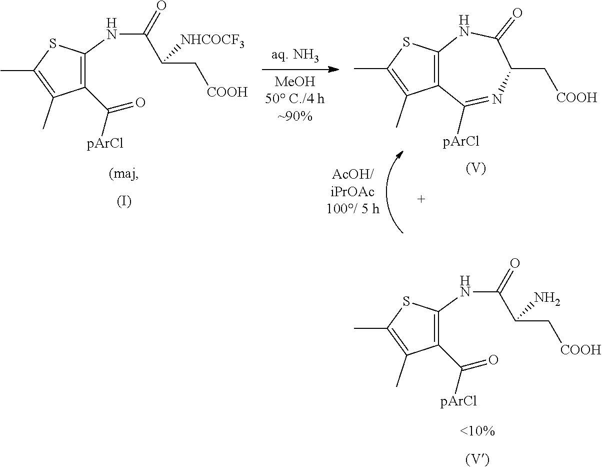

Amide (2.1 g, 4.4 mmol) was taken up in methanol (10 ml) and treated with 25% aqueous ammonia (4.7 ml, 31 mmol). The mixture was heated at 50° C. for 4 h and the resulting dark red solution was concentrated under reduced pressure. The residue was distributed between 10% aqueous sodium bicarbonate solution (25 ml) and ethylacetate (25 ml). The organic phase was separated and washed with additional bicarbonate solution (25 ml). The combined aqueous phases were acidified to ˜pH 4 with acetic acid (8.8 ml) then extracted with ethyl acetate (3×25 ml). The organic extract was washed with water (25 ml), dried over sodium sulphate and evaporated. Yield: 1.5 g orange foam (˜90%), HPLC 93%, 94:6 S/R.

The reaction repeated at ambient temperature for 16 h with 10 equiv. aqueous ammonia rendered an improved S/R ratio of 96:4. The reaction product consisted of ˜9:1 (V):(V′). When this material was treated with 2 equiv. acetic acid in isopropyl acetate and heated at 90° C. for ca. 5 h, residual (V′) cyclised to (V). However, with this method of cyclization, the S/R ratio declined to ˜80:20 when the deprotection step was conducted at RT or to ˜70:30 if the deprotection reaction was conducted at 50° C. Alternatively, isopropyl acetate could be used in place of ethyl acetate as extraction medium, acetic acid added and the extract treated directly as above.

### Example 4: [(S)-5-(4-Chlorophenyl)-6,7-dimethyl-2-oxo-2,3-dihydro-1H-thieno[2,3-e][1,4]diazepin-3-yl]-acetic acid methyl ester (VI)

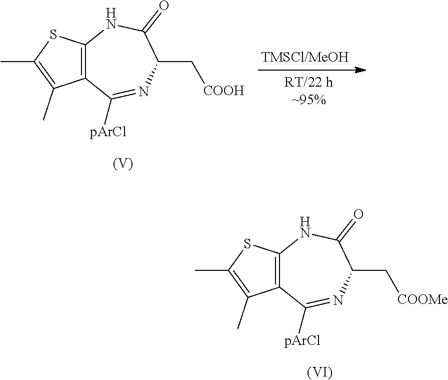

The acid (V) (27 mg, 75 μmol) in methanol (0.5 ml) was treated with trimethylsilyl chloride (0.29 μl, 225 μmol) and the solution was stirred at ambient temperature for 22 h. The solvent was removed under reduced pressure furnishing the product (VI) as the HCl-salt. Yield: 30 mg yellow crystalline solid (˜95%).

### Example 5: [(S)-4-(4-Chloro-phenyl)-2,3,9-trimethyl-6H-1-thia-5,7,8,9a-tetraaza-cyclopenta[e]azulen-6-yl]-acetic acid isobutyl ester (VI)

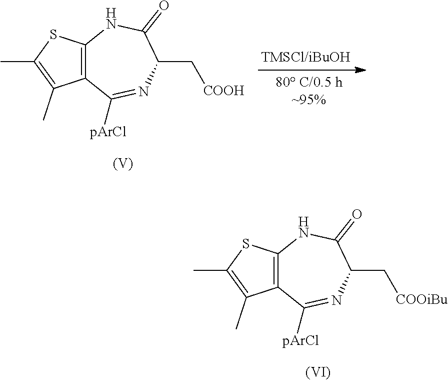

The acid (V) (1.0 g, 2.5 mmol, 94:6 S/R) in i-butanol (5 ml) was treated with trimethylsilyl chloride (0.64 ml, 5 mmol). The suspension was stirred at 80° C. for 0.5 h, creating a yellow solution. After removal of the solvent under reduced pressure, the residue was taken up in ethyl acetate (15 ml) and washed with saturated aqueous sodium bicarbonate (1M, 10 ml), and water (10 ml). The separated organic phase was dried over sodium sulphate, filtered and evaporated under reduced pressure. Yield: 0.97 g yellow foam (˜95%, 94:6 S/R).

Recrystalisation from 33% aqueous acetic acid rasied the S/R ratio to 99.6:0.4 (80-85% recovery)

### Example 6: [(S)-4-(4-Chloro-phenyl)-2,3,9-trimethyl-6H-1-thia-5,7,8,9a-tetraaza-cyclopenta[e]azulen-6-yl]-acetic acid isobutyl ester

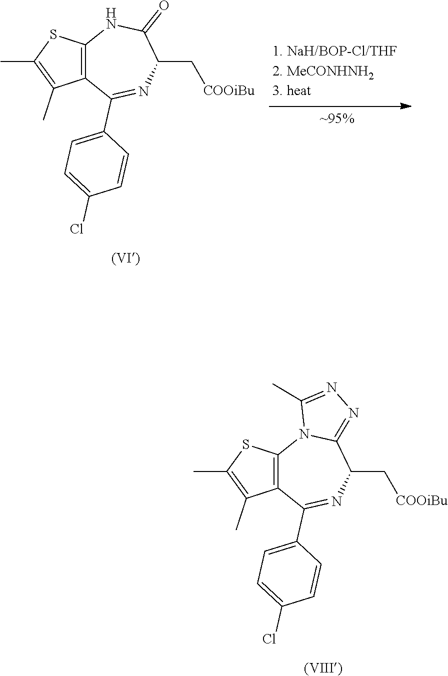

To a suspension of sodium hydride (0.085 g, 3.5 mmol) in dry tetrahydrofuran (10 ml), cooled to 0-5° C. was added a solution of the isobutyl ester (VI) in tetrahydrofuran (10 ml) over 0.1 h. A yellow suspension arose which was stirred at 5° C. for 0.1 h then treated with bis(2-oxo-3-oxazolidinyl)phosphinic chloride (0.89 g, 3.4 mmol) in one portion. The ensuing beige suspension was stirred at <5° C. for 2 h and acetylhydrazide (0.35 g, 4.5 mmol) was added. After stirring at RT for 3 h, the resulting thick orange suspension was heated at 65° C. for 2 h. The solvent was evaporated under reduced presure and the residue was taken up in ethyl acetate (10 ml) and washed twice with water (10 ml) which was back extracted with ethyl acetate. The combined organic phases were dried over sodium sulphate, filtered and evaporated under reduced pressure. Yield: 1.00 g tan foam (˜95%, 94:6 S/R).

### Example 7: [(S)-4-(4-Chloro-phenyl)-2,3,9-trimethyl-6H-1-thia-5,7,8,9a-tetraaza-cyclopenta[e]azulen-6-yl]-acetic acid (IX)

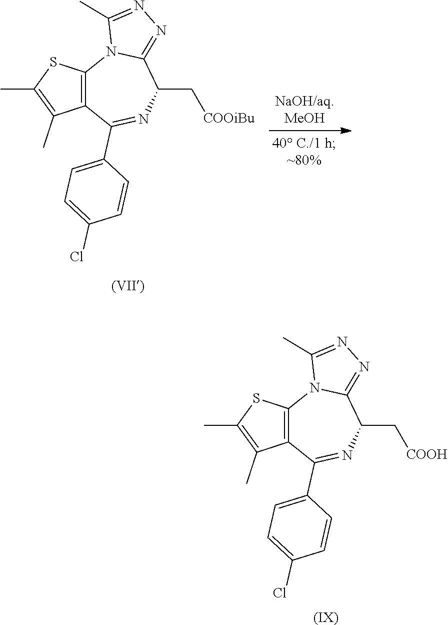

Crude isobutyl ester (VII′) (70 mg, 0.015 mmol) was taken up in methanol (0.7 ml) and a solution of sodium hydroxide (44 mg) in water (0.02 ml) was added. The brown solution was heated at 40° C. for 1 h. The reaction mixture was partitioned between ethyl acetate (10 ml) and water (5 ml). The aqueous phase was extracted with ethyl acetate (10 ml) & the organic phases with water (5 ml). The combined aqueous phases were treated with acetic acid (0.02 ml) to attain pH 5 and the product extracted into ethyl acetate (2×5 ml). The combined organic phases were washed twice with water (5 ml) then dried over sodium sulphate, filtered and evaporated under reduced pressure. Yield: 50 mg brown syrup (˜80%, 94:6 S/R).

Conversion of lactam (V) -->triazole (IX): effect on S/R ratio outcome
R=Me: 94:6→95:5 S/R (with coformation of lactam VII′)

### R=iPr: 94:6→86:14 S/R

### R=iBu: 94:6→94:6 S/R

### R=tBu: >99.5:0.5→>99.5:0.5 S/R (WO 2018/109053)

1. A compound of formula (I)

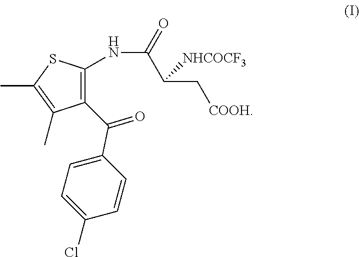

2. A process for the manufacture of a compound of formula (I) as defined in claim 1, comprising the reaction of a compound of formula (II) 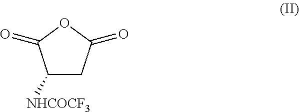
with a compound of formula (III)

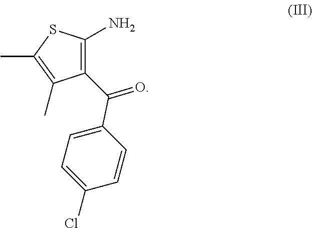 [SMILES: Cc1sc(N)c(C(=O)c2ccc(Cl)cc2)c1C]

3. A process according to claim 2, wherein the reaction is done in a solvent selected from acetone, trifluoroethanol, acetonitrile, tetrahydrofuran, methyltetrahydrofuran, ethyl acetate, dichloromethane, t-butylmethylether, toluene, benzotrifluoride and heptane.

4. A process according to claim 2, wherein the reaction is carried out in a non-polar solvent.

5. A process according to claim 4, wherein the solvent is dichloromethane or toluene, in particular dichloromethane.

6. A process according to claim 2, wherein the reaction is carried out at a temperature between 0° C. and room temperature.

7. A process according to claim 2, wherein the compound of formula (II) is prepared by the reaction of a compound of formula (IV) 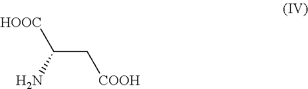
with trifluororoacetic anhydride.

8. A process for the manufacture of a compound of formula (V) 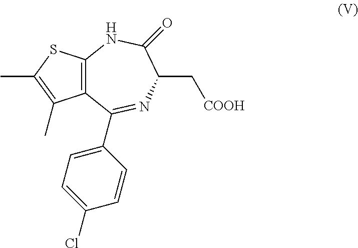 [SMILES: Cc1sc2c(c1C)C(c1ccc(Cl)cc1)=N[C@@H](CC(=O)O)C(=O)N2]
comprising the deprotection of the amino group —NHCOCF₃ of the compound of formula (I) into a primary amino group —NH₂ and formation of a ring to arrive at the compound of formula (V).

9. A process according to claim 8, wherein the deprotection of the amino group —NHCOCF₃ of the compound of formula (I) into a primary amino group —NH₂ is done by reaction of the compound of formula (I) with a base in an alcoholic medium.

10. A process according to claim 8, wherein the deprotection and ring formation are done at a temperature between room temperature and 100° C.

11. A process according to claim 8, further comprising separation of an uncyclized side product of formula (V′) 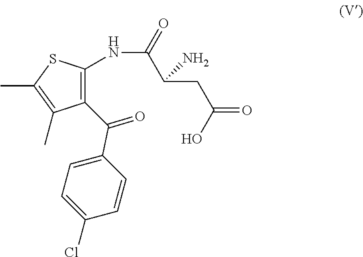 [SMILES: Cc1sc(NC(=O)[C@@H](N)CC(=O)O)c(C(=O)c2ccc(Cl)cc2)c1C]
obtained from the process of claim 8 from the reaction product and reaction of said uncyclized side product of formula (V′) with an acid to arrive at the compound of formula (V).

12. A process according to claim 11, wherein the acid is acetic acid, formic acid or a sulfonic acid.

13. A process according to claim 11, wherein the compound of formula (V′) is reacted with the acid in toluene or isopropyl acetate.

14. The use of a compound of formula (I) in the manufacture of a compound of formula (IX)

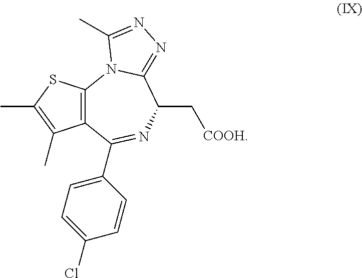 [SMILES: Cc1sc2c(c1C)C(c1ccc(Cl)cc1)=N[C@@H](CC(=O)O)c1nnc(C)n1-2]

15. A process for the manufacture of a compound of formula (IX) as defined in claim 14, comprising:
(a) reacting a compound of formula (V) with i-BuOH under acid catalysis to arrive at a compound of formula (VI′) 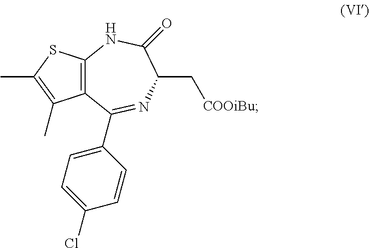
(b) reacting a compound of formula (VI′) with diethyl chlorophosphate, diphenyl chlorophosphate or bis(2-oxo-3-oxazolidinyl)phosphinic chloride and a base;
(c) reacting the product of step (b) with acetyl hydrazide followed by heating above room temperature to arrive at a compound of formula (VIII′) 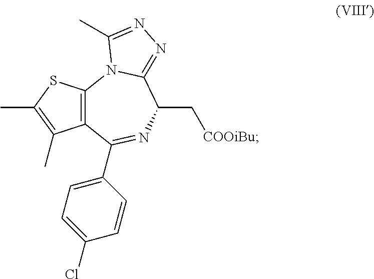
and
(d) deprotecting the carboxyl group of the compound of formula (VIII′) to arrive at the compound of formula (IX) as defined in claim 14.

16. (canceled)

17. A process according to claim 4, wherein the solvent is dichloromethane.

18. A process according to claim 11, wherein the acid is acetic acid.

19. A process according to claim 11, wherein the compound of formula (V′) is reacted with the acid in isopropyl acetate.
